(19) United States
(12) Patent Application Publication
(10) Pub. No.: US 2007/0252017 A1
(43) Pub. Date: Nov. 1, 2007

(54) Apparatus for Disseminating Volatile Liquids into an Atmosphere

(75) Inventors: Thomas McGee, Nyack, NY (US); Richard P. Sgaramella, Hoboken, NJ (US); Colin Brown, Bracknell Berkshire (GB); Guy Edward Naish, Oxfordshire (GB)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 10/577,633

(22) PCT Filed: Oct. 28, 2004
(86) PCT No.: PCT/CH04/00648

§ 371 Date: Mar. 5, 2007

Related U.S. Application Data
(60) Provisional application No. 60/518,793, filed on Nov. 10, 2003.

(51) Int. Cl. A61L 9/12

(52) U.S. Cl. 239/44

(57) ABSTRACT

An apparatus adapted to disseminate volatile liquid into an atmosphere from a reservoir, the transfer to atmosphere being achieved by means of a porous transfer member that transfers liquid from the reservoir to an evaporation surface. The evaporation surface is a capillary sheet substantially transversely from the transfer member, and being further characterized in that the material of the sheet is a plastics material having a Shore D hardness of from around 50 to 80 and a thickness of from 0.75-1.25 mm. These sheets make especially good liquid transfer contact with the transfer member.

This invention relates to apparatus for the disseminating of volatile liquids into an atmosphere.

One very common apparatus for disseminating a volatile liquid, such as a fragrance or an insecticide, into an atmosphere consists of a porous transfer member, such as a porous wick, that is in contact with a reservoir of volatile liquid. Liquid rises up this wick and evaporates into the atmosphere. This system has drawbacks, such as the low surface area for evaporation and the tendency for the wick to fractionate complex mixtures, such as fragrances, so that some components are disseminated earlier than others and the full effect of the fragrance is lost.

It has been proposed to overcome this disadvantage by using external capillaries, that is, capillary channels cut or moulded into a suitable substrate. One example is described in U.S. Pat. No. 4,913,350, in which an external capillary channel-containing member is inserted into a liquid. In another embodiment, described in copending UK Patent Application GB 0306449, there is fitted to a known transfer member a capillary sheet, that is, a sheet extending essentially perpendicularly from the transfer member and that comprises channels of capillary dimensions, to which volatile liquid can pass and travel along for evaporation. This sheet generally contacts the transfer member by means of a hole in the sheet through which the transfer member protrudes and within which it fits snugly, at least some of these channels contacting the transfer member such that liquid can transfer from the member to the sheet (“liquid transfer contact”).

Although this technology offers significant advantages over the porous wicks of the art, these advantages have never been completely realized. It has now been found that it is possible to obtain the full benefits of the technology by adherence to certain fundamental parameters. The invention therefore provides an apparatus adapted to disseminate volatile liquid into an atmosphere from a reservoir, the transfer to atmosphere being achieved by means of a porous transfer member that transfers liquid from the reservoir to an evaporation surface, the evaporation surface being a capillary sheet in liquid transfer contact with and extending substantially transversely from the transfer member, and being further characterized in that the material of the sheet is a plastics material having a Shore D hardness of from 50 to 80 and a thickness of from 0.75-1.25 mm.

The capillary sheet and transfer member are separate components able to be put together when necessary. Typical transfer members include porous wicks of plastics, graphite and ceramics, made by any suitable method, for example, by extrusion or sintering. A preferred material is a porous plastic, such as polyester. The transfer member may be of any suitable shape or construction. Some examples of suitable configurations include;
the transfer member being slightly frusto-conical in shape, narrower end farthest from the reservoir; this enables a capillary sheet with a circular aperture to be easily slipped on and fitted to the transfer member;
the provision in the transfer member of a suitable locating orifice for a capillary sheet, for example, an annular groove or a slot for a matching tab in the capillary diffusion member, to allow for easy fitting.

A combination of all or any of these elements may be used. In addition, other constructions that are not mentioned here but which lie within the skill of the art are also included.

The plastics material may be chosen for any suitable plastics material having the desired hardness properties Shore D hardness is a very well known property in the plastics industry and its measurement is common. It is a surprising feature of this invention that this parameter should have any effect on the efficacy of an evaporation surface, yet it has been found that this has a marked influence. The desired Shore D hardnesses are quite soft by the standards of the plastics industry. Naturally the plastics material should also be suitably inert with respect to any of the materials in the liquid. It should also be a material that permits the formation of capillary channels by any suitable means, for example, by engraving or injection moulding. Injection moulding is the preferred method of forming a capillary sheet. Capillary channels are typically V-shaped channels whose typical dimensions are 0.1-0.5 mm wide, 0.1-0.5 mm deep with the “V” angle of the channel being 10-25 degrees. The skilled person will readily appreciate what kind of plastics material is suitable for any use.

In a preferred embodiment of the invention, the plastics materials for use in the making of capillary sheets suitable for use in this invention have a surface energy of from 15-50 dyne/cm. The surface energy of a plastics material is dependent upon its molecular structure and is a measure of the ability of a surface to be wetted. The more inert is a plastics material chemically, the lower is its surface energy. Thus, materials such as polyethylene, polypropylene and PTFE have low surface energies, whereas the plastics with more polar groups have higher surface energies. Preferably the surface energy lies in the range of from 30-45 dynes/cm.

The apparatus is generally supplied with the capillary sheet dismounted and a protective cap over the porous member. The apparatus is put into service by removing the cap and placing the capillary sheet on the transfer member, such that it is in liquid transfer contact with the transfer member. This can be assured by use, for example, of one of the attachment means hereinabove described.

The invention is suitable for use with any volatile liquid that may be disseminated into an atmosphere by means of a porous wick. Commercial materials of this type, especially the fragrances, are generally carefully formulated proprietary compositions containing a plurality of ingredients, the precise nature of such compositions being kept confidential. However, whatever the nature of these compositions, it is a feature of this invention that they are much more effectively disseminated by the apparatus according to this invention than by a wick alone.

The invention therefore additionally provides a method of disseminating a volatile liquid into an atmosphere by means of its absorption in and travel along an essentially cylindrical porous wick and then along an evaporation surface extending substantially transversely from the wick and in liquid transfer contact therewith, the evaporation surface comprising a capillary sheet of a plastics material having a Shore D hardness of from 50-80 and a thickness of from 0.75-1.25 mm.

The invention is further described with reference to the following non-limiting examples.

### EXAMPLE 1

1.00 mm thick external capillary sheets were prepared from the materials in the following table and the Shore hardness measured. This was done by means of a Shore (Durometer) test according to ASTM D2240 00. The apparatus used was a Mitutoyo Hardmatic HH-337-01. An indenter is pressed in the plastic and this forces the needle of the gauge round and the indicator records maximum value. This is the Shore D Hardness. The individual sheets comprised central holes that permitted them to be mounted on frusto-conical wicks by simply placing them on the wicks.

| | | Hardness |
|---|---|---|
| | | (Measured) |
| Trade Name | Material Type | (Shore D) |
| BOREALIS* MG 9641-R | Polyethylene PE (HDPE) | 56 |
| SAN* 386R/774 | Styrene Acrylonitrile | 87 |
| | Copolymer SAN | |
| EXACT* 8210 | Octene-1 Plastomer | 44 |
| POLYAC* PA-758 | Acrylonitrile-butadiene- | 81 |
| | styrene ABS | |
| IUPITAL* F40-03 | Polyoxymethylene POM | 80 |
| | (Acetal) | |
| LUPOY* GP5001 A-F | PC/ABS | 80 |
| IPETHENE* 320 | Polyethylene PE (LDPE) | 45 |
| RADIATER* E AX1 100 | Polyethylene terepthalate | 79 |
| | PETG | |
| LL6201 | Polyethylene PE (LLDPE) | 47 |
| ESCORENE* VL02020 | Ethylene Vinyl Acetate | 41 |
| | EVA | |
| PS 146L | Polystyrene PS | 83 |
| IUPILON* S3000 | Polycarbonate PC | 84 |
| PP 7075 L1 | Polypropylene PP | 55 |
| ITOCHU* H2O2 | Polyethylene terepthalate | 74 |
| | PETG | |
| IOTEK* 8020 | Ionomer (sodium) | 62 |

*Trade marks

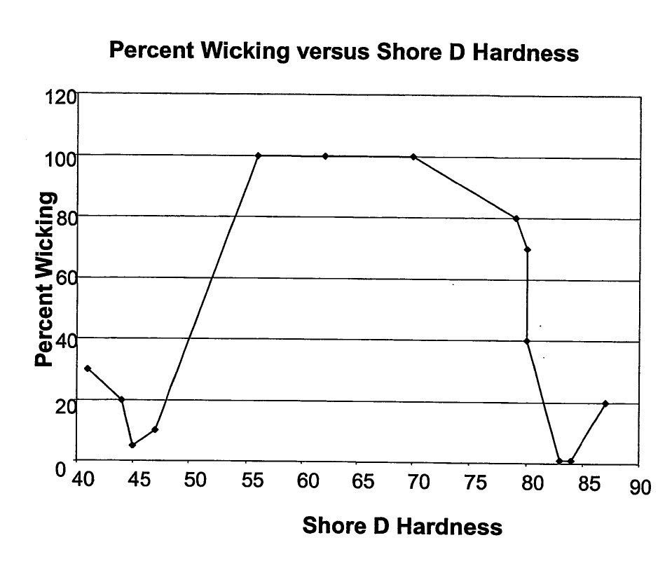

Frusto-conical wicks of polyester (ex Micropore) were placed in a container containing a vanilla fragrance formulation used for air freshening applications (this fragrance has the advantage of being easily visible). They were allowed to equilibrate overnight. The external capillary sheets were pushed on to a frusto-conical wick until firm contact between sheet and wick (suitable to ensure liquid transfer) was made. The percentage of the capillaries in which liquid was present was assessed visually assessed after 6 minutes (0% for no transfer from wick to the capillary, 100% for the presence of liquid in all capillaries and liquid has traveled to end of all capillaries). The results are shown on the graph of FIG. 1.

The graph shows that, for optimum liquid transfer, the material has to have a Shore D hardness of between 50 and 80.

### EXAMPLE 2

Two frusto-conical wicks were placed in individual containers containing the fragrance formulation of Example 1. They were allowed to equilibrate overnight. To one of these wicks was added a polypropylene capillary sheet having a thickness of 1 mm and a Shore hardness of 55, to the other an otherwise identical sheet having a thickness of 1.5 mm. The percentage of each sheet wetted by the liquid is shown in the following table.

| Thickness of External Capillary Sheet | % capillary sheet wetted |
|---|---|
| 1.5 mm | 25% |
| 1.0 mm | 100% |

Hence the capillary thickness specification for direct liquid transfer as described in U.S. Pat. No. 4,913,350 is not suitable for use in a hybrid wick system as it does not give optimum liquid transfer contact. The ideal thickness is 1.25 mm maximum. The lower limit of thickness is 0.75 mm set by the limit that good capillaries formed in a suitable production process such as injection molding.

1. An apparatus adapted to disseminate volatile liquid into an atmosphere from a reservoir, the transfer to atmosphere being achieved by means of a porous transfer member that transfers liquid from the reservoir to an evaporation surface, the evaporation surface being a capillary sheet in liquid transfer contact with and extending substantially transversely from the transfer member, and being further characterized in that the material of the sheet is a plastics material having a Shore D hardness of from around 50 to 80 and a thickness of from 0.75-1.25 mm.

2. An apparatus according to claim 1, in which the arrangement of transfer member and capillary sheet is selected from the group consisting of:
(i) a transfer member that is slightly frusto-conical in shape, narrower end farthest from the reservoir, on to which a capillary sheet having a circular aperture is slipped and fitted;
(ii) a transfer member comprising a suitable locating orifice into which a capillary sheet may be fitted.

3. An apparatus according to claim 1, in which the plastics materials of the capillary sheet has a surface energy of from 15-50 dyne/cm.

4. An apparatus according to claim 2, in which the surface energy lies in the range of from 30-45 dynes/cm.

5. An apparatus according to claim 1, in which the capillary channels are V-shaped channels that are from 0.1-0.5 mm wide, from 0.1-0.5 mm deep, and that have a “V” angle of the channel of from 10-25 degrees.

6. A method of disseminating a volatile liquid into an atmosphere by means of its absorption in and travel along an essentially cylindrical porous wick and then along an evaporation surface extending substantially transversely from the wick and in liquid transfer contact therewith, the evaporation surface comprising a capillary sheet of a plastics material having a Shore D hardness of from 50-80 and a thickness of from 0.75-1.25 mm.
